(19) United States
(12) Patent Application Publication
(10) Pub. No.: US 2001/0033859 A1
(43) Pub. Date: Oct. 25, 2001

(54) Transdermal therapeutic system for delivery of dofetilide

(76) Inventor: Torsten Selzer, Neuwied (DE)

Correspondence Address: William F. Lawrence, Esq., c/o FROMMER, LAWRENCE & HAUG LLP, 745 Fifth Avenue, New York, NY, 10151, US

(21) Appl. No.: 09/836,707

(22) Filed: Apr. 17, 2001

(30) Foreign Application Priority Data
Apr. 18, 2000 (DE) 100 19 067.7

(51) Int. Cl. A61K 9/70

(52) U.S. Cl. 424/447

(57) ABSTRACT

The invention relates to a dofetilide-containing transdermal therapeutic system in plaster form with a backing layer which is impermeable to active ingredient, with an active ingredient reservoir which is connected thereto, with a contact adhesive layer and with a protective layer which can be detached before application.

The invention additionally relates to the use of such transdermal therapeutic systems for medical use for various indications.

The invention relates to a transdermal therapeutic system which makes it possible to deliver dofetilide N-[4-[2-[methyl[2-[4-[(methylsulfonyl)amino]phenoxy] ethyl]amino]ethyl]phenyl]-methanesulfonamide onto and through the skin. The formula of dofetilide is

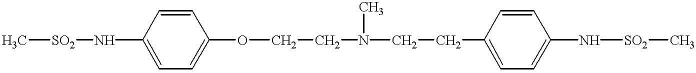

The invention also relates to the use of such a system for a wide variety of medical indications.

### BACKGROUND OF THE INVENTION

Transdermal therapeutic systems (TTS) are dosage forms which are applied to the skin and are intended to make a drug substance systemically available. TTS may increase the therapeutic value of administration of a drug substance by ensuring constant delivery of the drug substance to the vascular system over a prolonged period. Problems such as gastrointestinal intolerance, low enteral absorption, metabolism during the first pass through the bowel and liver, and high frequency of administration associated with low half-lives can thus be avoided.

Prior art TTS consist of a backing layer which is impermeable to drug substance, of a reservoir layer containing drug substance, where appropriate of a control membrane, and of a contact adhesive layer for attachment to the skin, it being possible for the latter to be identical to the reservoir layer containing drug substance, and of a protective layer which is to be removed before application and is likewise impermeable to drug substance. The reservoir layer containing drug substance consists of drug substance and excipients such as plasticizers, tackifiers, solubilizers, stabilizers, fillers, carriers and permeation promoters. The pharmaceutically acceptable substances suitable for this purpose are known to the skilled worker.

Dofetilide is an oral class III antiarrhythmic which is employed for conversion and maintenance of a normal sinus rhythm in patients with atrial fibrillation and flutter. The disadvantages of dofetilide derive from the pharmacokinetics: on oral administration, dofetilide is subject to pronounced metabolism during the first pass through the bowel and liver and has a short plasma half-life.

### DESCRIPTION OF THE INVENTION

It was an object of the present invention to provide a dosage form for the active ingredient dofetilide which avoids said disadvantages.

The object is achieved by a transdermal dosage form in plaster form which makes it possible for the active ingredient flux in vivo to be sufficiently high and which can be produced cost-efficiently by conventional production processes. Because of its properties, namely the high melting point (above 150° C.) and molecular weight (442 g/mol), a partition coefficient of about 1 and the two sulfonanilide groups which are present and which are prone to hydrogen bonding, which has been shown by experience to make transdermal permeation difficult, it was not to be expected that dofetilide could be used in a satisfactory way in the form of transdermal systems.

The invention thus relates to a dofetilide-containing transdermal therapeutic system in plaster form with a backing layer impermeable to active ingredient, with an active ingredient reservoir connected thereto, with a contact adhesive layer and with a protective layer which can be detached before application.

The advantages arise, as described above, because of the constant delivery of the active ingredient over the application period. Rapid elimination of dofetilide is thus compensated by continual replenishment from the transdermal system. In addition, the problem of the low oral availability is avoided.

The TTS of the invention can be employed both in the form of matrix systems and in the form of membrane systems. The term “matrix systems” includes not only those systems in which the active ingredient is dissolved in a synthetic resin or synthetic material matrix and is delivered therefrom, but also those in which the active ingredient is adsorbed in a fiber material such as a woven or nonwoven cotton fabric. It is immaterial in this connection, especially for matrix systems, which polymers, resins and possibly other additives are employed as long as the substances which come into contact with the skin are compatible with skin, and the formulation is suitable for delivering the active ingredient dofetilide onto the skin.

In the simplest case, dofetilide can be in the form of a coarse, colloidal or molecular dispersion in a solution of basic polymers, and the mixture can then be coated onto a suitable substrate—usually a thermoplastic sheet provided with a silicone layer—and, after evaporation of the solvent contents, be covered with a further sheet which represents the subsequent backing of the TTS. TTS are obtained from such a laminate by punching out sheet-like structures in the required geometric shape. Suitable basic polymers for the system of the invention 4 are, for the matrix and the contact adhesive, polymers based on acrylic and/or methacrylic acid and esters thereof, isobutylene, ethylene/vinyl acetate and/or natural and synthetic rubbers such as styrene/diene copolymers, for example styrene/butadiene block copolymers, or isoprene block copolymers, acrylonitrile/butadiene rubbers, butyl rubber or neoprene rubber or hot melt adhesives. Suitable contact adhesives are also those based on silicones. This list is not complete but does reveal the broad applicability of the principle of the invention.

A preferred embodiment of the invention comprises the dofetilide being present in the TTS in combination with a solvent, preferably in the dissolved state, in which case the formulation should, where appropriate, comprise a solubilizer. Examples of solubilizers are polyhydric alcohols such as 1,2-propanediol, the various butanediols, glycerol, polyethylene glycol 400, also tetrahydrofurfuryl alcohol, diethylene glycol monoethyl ether, diethyltoluamide, monoisopropylideneglycerol etc. 1,2-Propanediol is preferred. It has proved to be advantageous for the content of solubilizer to be between 1 and 50% by weight, preferably between 5 and 35% by weight, based on the complete TTS. Some of these solubilizers such as 1,2-propanediol can also act as permeation-promoting substances.

In order to achieve a high flux with matrix systems it has proved to be particularly advantageous to add the permeation-promoting substances in an amount of from 0.1 to 25% by weight, preferably between 1 and 10% by weight, based on the total weight of the matrix. Examples thereof are fatty alcohols such as decanol, dodecanol, fatty acids such as oleic acid, myristic acid, polyoxyethylene fatty alcohol ethers. Of the latter, those preferably employed are polyoxyethylene lauryl ethers (Brij®). Also suitable are polyoxyethylene fatty acid esters and fatty acid esters such as sorbitan monolaurate or esters of long-chain fatty acids with methyl, ethyl or isopropyl alcohol or esters of fatty alcohols with acidic acid or lactic acid, and substances such as oleic acid diethanolamide.

The TTS of the invention may also have a layer structure with two or more matrix layers which comprise, for example, polymeric components from the group of substituted celluloses, preferably methyl- and ethylcelluloses. It is moreover possible for the individual matrix layers to comprise different concentrations of active ingredient, permeation-promoting agents and solubilizers. Depending on the intended purpose of use, the concentrations in these layers may differ so that they decrease or increase from the inner layer to the layer on the skin side depending on whether a particular long-term action or an initial action is desired. A further possibility is for the individual matrix layers to consist of different contact adhesives, and to comprise conventional plasticizers in a concentration of up to 30% by weight, preferably 5 to 20% by weight, from the group of hydrocarbons, alcohols, carboxylic acids and derivatives thereof, ethers, esters or amines. In order to make it possible to control release, unless this is brought about by other mechanisms, the reservoir can also be provided with a control membrane which controls delivery of the active ingredient onto the skin.

There are likewise various possibilities for attaching the transdermal system to the skin. For example, the matrix may itself consist of a contact adhesive and thus produce the connection to the skin. However, it is also conceivable in the case of systems with a control membrane for the TTS to be produced so that the attachment to the skin is brought about by an adhesive margin which is not in contact with the area through which the active ingredient is delivered onto the skin, that is to say it is not in contact with the control membrane.

The TTS of the invention additionally contains one backing layer impermeable to the active ingredient, and a detachable protective layer or pull-off sheet which is likewise impermeable to the active ingredient. Particularly suitable as backing layer are polyesters distinguished by special strengths, such as polyethylene terephthalate and polybutylene terephthalate, but also almost any other synthetic materials which are compatible with skin, such as polyvinyl chloride, ethylene/vinyl acetate copolymers, polyvinyl acetate, polyethylene, polypropylene, polyurethanes, cellulose derivatives and many others. It is possible in the particular case for the backing layer to be provided with an additional layer on top, for example by vapor deposition of metals, especially aluminum. It is possible to use the same materials for the detachable protective layer as for the backing layer, provided it is given a detachable finish through a suitable surface treatment such as siliconization. However, it is also possible to use other detachable protective layers such as polytetrafluoroethylene-treated paper or cellophane (cellulose hydrate).

The active ingredient may also be present in a bag-like reservoir which is filled with a synthetic material matrix which is capable of flowing, for example highly viscous or semisolid, or a solution thereof, which contains the active ingredient. It is particularly advantageous if the active ingredient reservoir contains a gel former. The reverse side of the bag facing away from the skin must in this case be impermeable to active ingredient, and the side facing the skin must be permeable to active ingredient. It is possible where appropriate for a membrane which is permeable to active ingredient to take over control of the release of active ingredient.

In order to achieve a high rate of release, it is preferred to provide a concentration of active ingredient which is as high as possible in the active ingredient-containing layers, although it must be noted that if the concentrations are too high the physical stability may be impaired. The concentrations of active ingredient used in the TTS of the invention are therefore in the range from 0.1 to 50% by weight, in particular from 1 to 10% by weight, based on the total weight of the active ingredient-containing layers.

The TTS of the invention are suitable for medical use in a wide variety of indications such as for the treatment of cardiac dysrhythmias, heart failure, peripheral blood flow disturbances, elevated blood pressure and for preventing attacks of angina pectoris. They are suitable for administering dofetilide with a duration of action of from about one day up to seven days, depending on the content of active ingredient and design of the control mechanism.

### EXAMPLE

The TTS of the invention can be produced as follows, for example: 50 g of dofetilide and 20 g of a suitable permeation-promoting substance (for example Brij®30) are dissolved in 200 g of 1,2-propanediol. This solution is added with the aid of a suitable stirring apparatus to the silicone adhesive 4301 from Dow Corning (USA) and dispersed so that a liquid/liquid dispersion of maximum homogeneity is produced. This dispersion is coated homogeneously with a suitable device onto a support sheet, for example made of polyethylene terephthalate. Subsequently, by controlled drying, the solvent of the silicone adhesive and any propanediol contents are removed. The laminate obtained in this way is then laminated with another sheet of polyethylene terephthalate. Finally, TTS with a defined area are punched out and packed in a suitable packaging.

1. A dofetilide-containing transdermal therapeutic system in plaster form, comprising a backing layer which is impermeable to active ingredient, an active ingredient reservoir which is connected thereto as matrix or membrane system, a contact adhesive layer and a protective layer which can be detached before application, where the active ingredient concentration is in the range from 0.1 to 50% by weight, based on the total weight of the active ingredient-containing layers.

2. A transdermal therapeutic system as claimed in claim 1, wherein the dofetilide is present in combination with a solvent, preferably one which additionally has a permeation-promoting action, in particular in the dissolved state.

3. A transdermal therapeutic system as claimed in claim 2, wherein the solvent is present in an amount of from 1 to 50, preferably 5 to 35,% by weight.

4. A transdermal therapeutic system as claimed in one or more of claims 1 to 3, which is in the form of a matrix system and comprises a permeation-promoting substance, expediently in an amount of from 0.1 to 25% by weight, preferably 1 to 10% by weight, based on the total weight of the matrix.

5. A transdermal therapeutic system as claimed in one or more of claims 1 to 4, wherein the active ingredient concentration is in the range from 0.1 to 10% by weight.

6. A transdermal therapeutic system as claimed in one or more of claims 1 to 5, which has a layered structure with at least two polymer matrix layers, preferably differing in the dofetilide concentration.

7. A transdermal therapeutic system as claimed in one or more of claims 1 to 6, wherein the basic polymers for the matrix and the contact adhesive are polymers based on acrylic and/or methacrylic acid and esters thereof, isobutylene, ethylene/vinyl acetate or are styrene/butadiene block copolymers or isoprene block copolymers and/or wherein the contact adhesives are silicone-based substances.

8. The use of a transdermal therapeutic system as claimed in one or more of claims 1 to 7 for the treatment of cardiac dysrhythmias and/or of heart failure and/or for preventing attacks of angina pectoris.

9. The use of the transdermal therapeutic system as claimed in one or more of claims 1 to 7 for the treatment of peripheral blood flow disturbances.

10. The use of the transdermal therapeutic system as claimed in one or more of claims 1 to 7 for the treatment of elevated blood pressure.
